(19) United States
(12) Patent Application Publication
(10) Pub. No.: US 2020/0239693 A1
(43) Pub. Date: Jul. 30, 2020

(54) QUINOPHTHALONE COMPOUND

(71) Applicant: DIC Corporation, Tokyo (JP)

(72) Inventors: Tatsuya Shigehiro, Sakura-shi (JP); Hitoshi Kondo, Sakura-shi (JP); Kengo Yasui, Sakura-shi (JP)

(73) Assignee: DIC Corporation, Tokyo (JP)

(21) Appl. No.: 16/490,274

(22) PCT Filed: Feb. 20, 2018
(86) PCT No.: PCT/JP2018/005875

§ 371 Date: Aug. 30, 2019

(30) Foreign Application Priority Data
Mar. 1, 2017 (JP) 2017-038267

(51) Int. Cl. C09B 25/00; C07D 401/14; G02B 5/22

(52) U.S. Cl. CPC C09B 25/00; G02B 5/223; C07D 401/14

(57) ABSTRACT

It is an object of the present invention to provide a novel yellow pigment having excellent brightness and coloring power. The object is achieved by providing a quinophthalone compound represented by the following Formula (1):

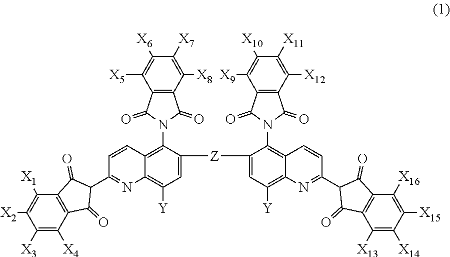

(in Formula (1), X₁ to X₁₆ are each independently a hydrogen atom or a halogen atom, Y is a hydrogen atom or a halogen atom, and Z is a lower alkylene group). Furthermore, a coloring agent containing a quinophthalone compound represented by Formula (1) and a coloring composition containing the coloring agent are provided.

### TECHNICAL FIELD

The present invention relates to a novel quinophthalone compound.

### BACKGROUND ART

PTLs 1 and 2 disclose bis-quinophthalone compounds having certain structures. PTL 3 discloses a quinophthalone compound having a certain structure. However, PTLs 1, 2, and 3 do not disclose compounds represented by the following Formula (1) of the present invention.

### CITATION LIST

### Patent Literature

PTL 1: Japanese Examined Patent Application Publication No. 48-32765

PTL 2: WO 2013/098836

PTL 3: Japanese Unexamined Patent Application Publication No. 53-228

### SUMMARY OF INVENTION

### Technical Problem

Currently, coloring compositions are used in various fields, and examples of the use include printing inks, paints, resin-coloring agents, fiber-coloring agents, and information-recording color materials (color materials to be used in color filter, toner, ink jet, etc.). The coloring matter that is used in coloring compositions is mainly classified into pigments and dyes and is required to have performances such as color characteristics (coloring power and clearness) and resistance (weather resistance, light resistance, heat resistance, and solvent resistance). In general, a pigment develops a color in the particulate state (aggregate of primary particles), whereas a dye develops a color in the molecular state. Accordingly, although pigments are generally superior to dyes in resistance, many of pigments are inferior to dyes in coloring power and clearness. From such a background, pigments having high coloring power and high saturation are demanded. In particular, organic pigments, which are regarded as excellent in coloring power, are especially attracting attention.

In the organic pigments, for example, the number of yellow pigments registered in the Colour Index is large next to that of red pigments, and a large number of novel pigments have been actively developed. Examples of the yellow organic pigment that is actually used include, in printing ink application, C.I. Pigment Yellows 3, 12, and 74; in paint application, C.I. Pigment Yellows 74, 83, 109, and 110; and in color filter application, C.I. Pigment Yellows 129, 138, 150, and 185. However, since the basic structures thereof are, for example, mainly azo, azomethine, isoindoline, and isoindolinone, the structural variations of yellow organic pigments are still insufficient for responding to required various applications.

In particular, the color filters for liquid crystal displays or pigments used in such color filters are required to have characteristics different from those in known general-purpose uses. Specifically, for example, “high brightness” that can reduce the backlight power consumption and “high coloring power” that enables a reduction in the thickness of color filter and high color reproduction are required. However, the current state is that there is not a pigment that meets all these requirements in existing yellow pigments for color filters.

Here, a color filter consists of red pixels (R), green pixels (G), and blue pixels (B), and yellow pigments are used for toning green pixels in many cases. Among yellow pigments, the amount used of C.I. Pigment Yellow 138 is the highest. However, C.I. Pigment Yellow 138 is poor in coloring power and is not practical as a color standard that is required to have high color reproducibility. Accordingly, in the color standard of high color reproducibility, C.I. Pigment Yellow 150 is currently used as the yellow pigment. However, the brightness and the coloring power of C.I. Pigment Yellow 150 are both also insufficient. Accordingly, it is desired to create a novel yellow pigment having excellent brightness and coloring power.

### Solution to Problem

The present inventors have diligently studied for solving the above-described problems and as a result, have found that more selective absorption and transmission are exhibited by dimerizing a quinophthalone skeleton. Furthermore, the inventors have also studied dimerization methods and have found that an excessive reddish tone can be prevented by cutting the conjugate using a lower alkylene chain as a spacer, instead of simply adopting a direct bonding. In addition, dispersibility was improved by polyhalogenation and introduction of an imide structure.

The present inventors have found based on the results of the studies that the above-mentioned problems can be solved by a compound represented by the following Formula (1), and accomplished the present invention. That is, the present invention relates to a quinophthalone compound (hereinafter, may be referred to as “the inventive compound”) represented by Formula (1):

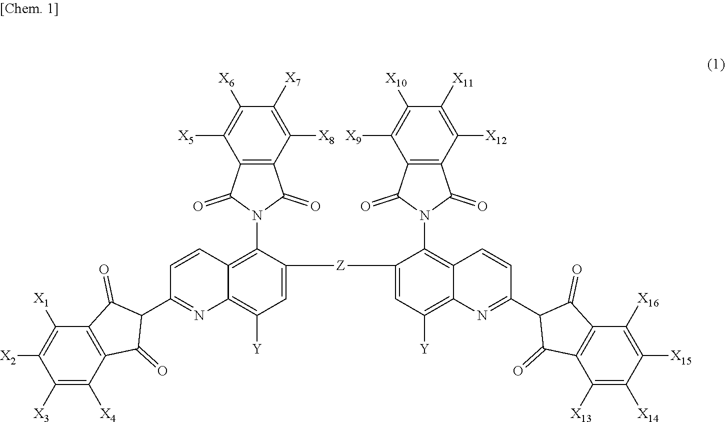

(in Formula (1), X₁ to X₁₆ are each independently a hydrogen atom or a halogen atom, Y is a hydrogen atom or a halogen atom, and Z is a lower alkylene group).

### Advantageous Effects of Invention

The inventive compounds have excellent brightness and coloring power. In particular, in the color standard for high color reproducibility in application to color filters, the inventive compound has better brightness than that of an existing yellow pigment (C.I. Pigment Yellow 150) and higher coloring power than that of the yellow pigment.

### DESCRIPTION OF EMBODIMENTS

Examples of the halogen atom in Formula (1) include fluorine, chlorine, bromine, and iodine atoms. In particular, a fluorine atom, a chlorine atom, and a bromine atom are preferred, and a chlorine atom is more preferred.

Examples of the lower alkylene group in Formula (1) include C1-C3 alkylenes such as methylene, ethylene, and propylene. In particular, a case of methylene is more preferred.

The quinophthalone compound represented by Formula (1) exists as tautomers having structures represented by, for example, Formula (1-i) or Formula (1-ii), and these tautomers are also encompassed in the present invention.

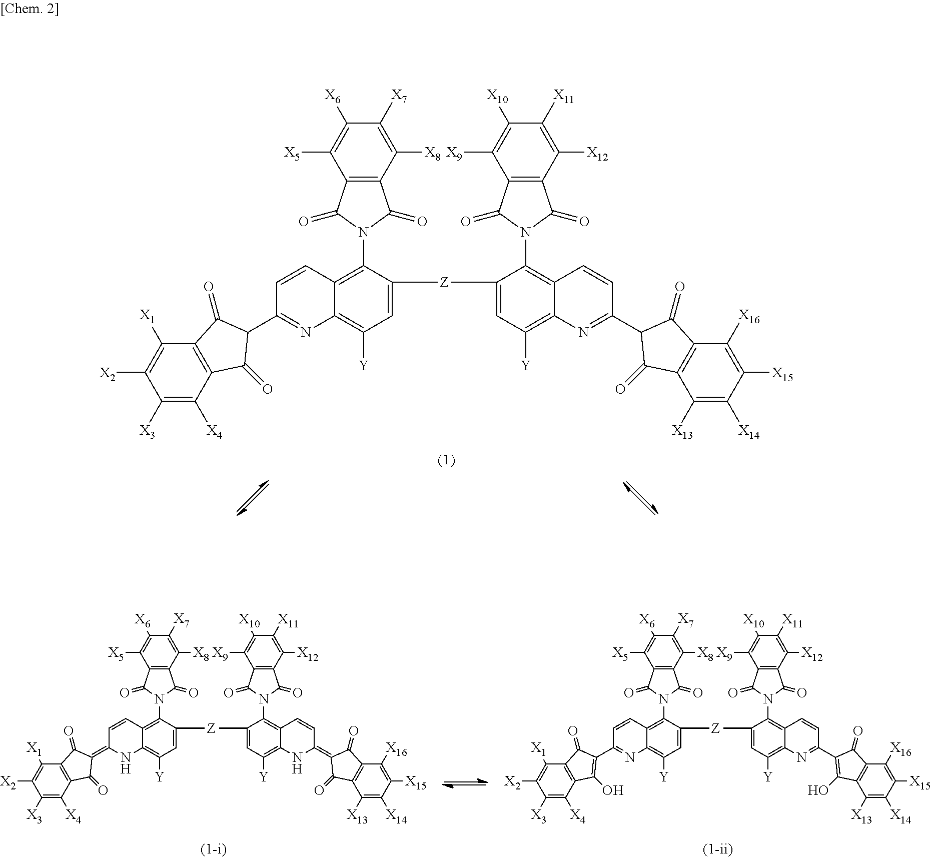

In Formulae (1-i) and (1-ii), X₁ to X₁₆, Y, and Z are as described above.

Examples of the quinophthalone compound of the present invention are shown below, but the present invention is not limited thereto.

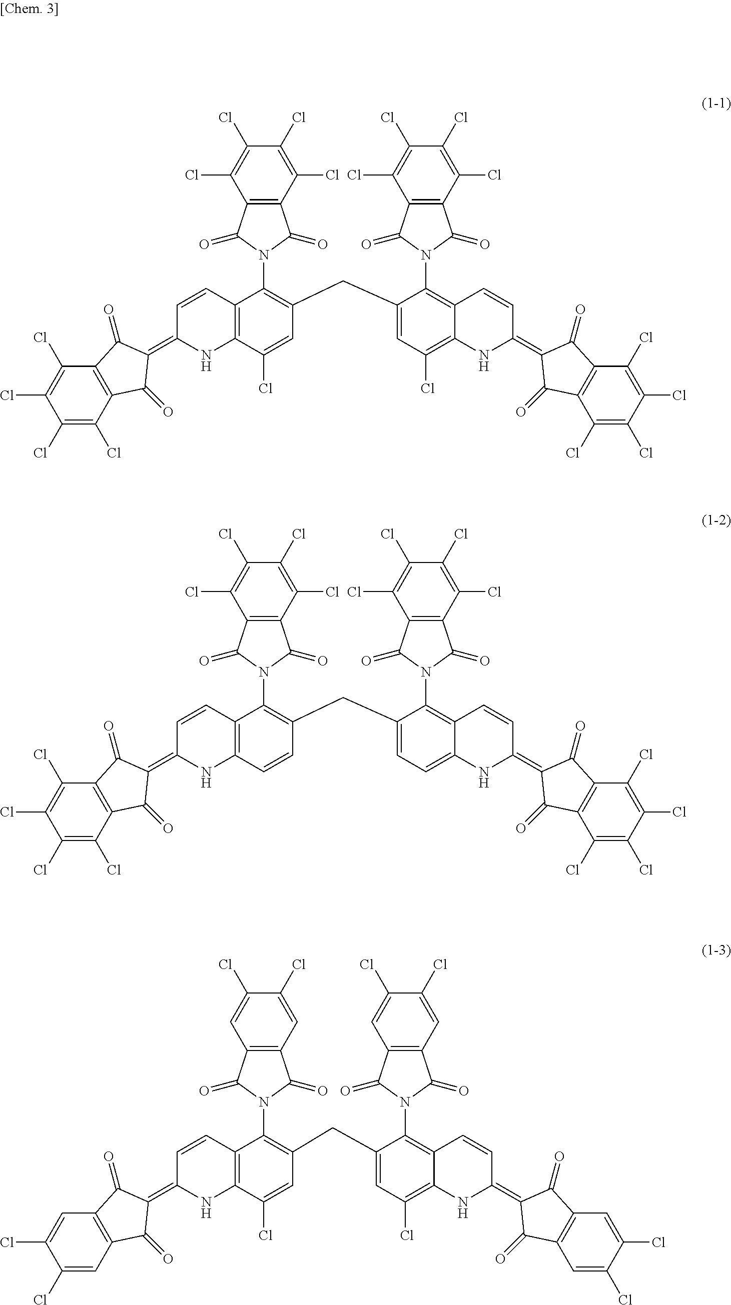

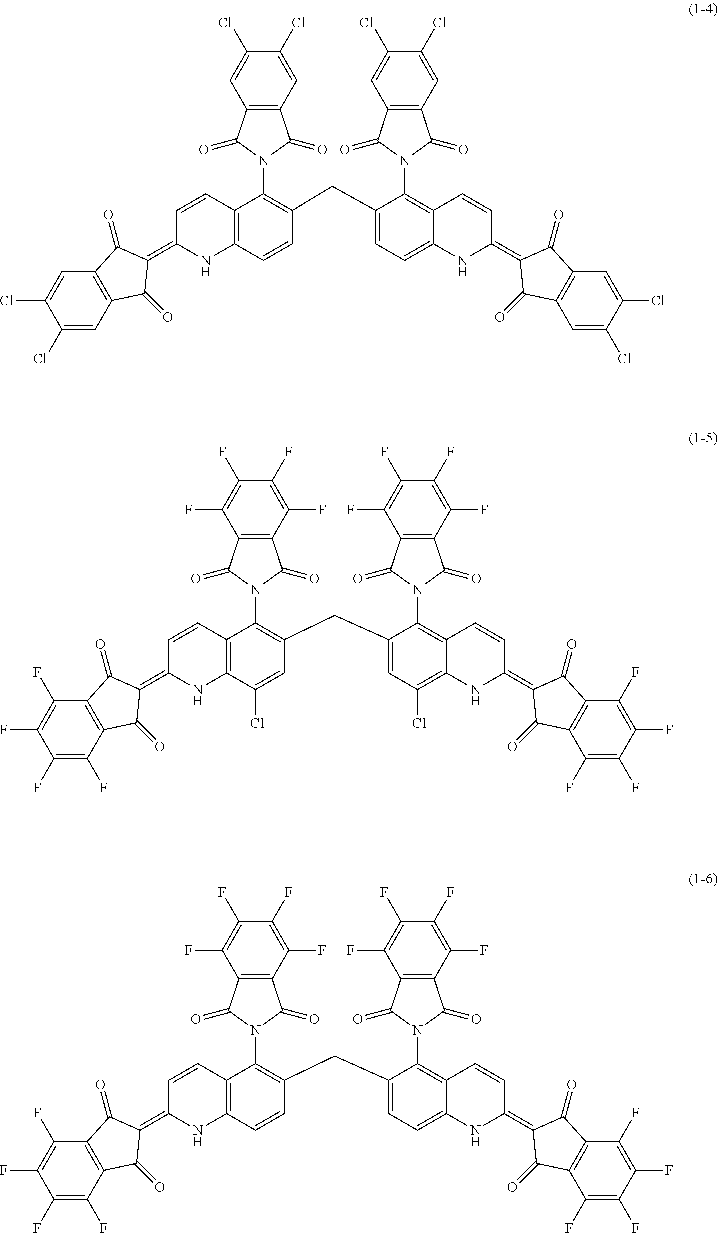 [SMILES: O=C1C(=C2C=Cc3c(ccc(Cc4ccc5c(c4N4C(=O)c6cc(Cl)c(Cl)cc6C4=O)C=CC(=C4C(=O)c6cc(Cl)c(Cl)cc6C4=O)N5)c3N3C(=O)c4cc(Cl)c(Cl)cc4C3=O)N2)C(=O)c2cc(Cl)c(Cl)cc21 ; O=C1C(=C2C=Cc3c(c(Cl)cc(Cc4cc(Cl)c5c(c4N4C(=O)c6c(F)c(F)c(F)c(F)c6C4=O)C=CC(=C4C(=O)c6c(F)c(F)c(F)c(F)c6C4=O)N5)c3N3C(=O)c4c(F)c(F)c(F)c(F)c4C3=O)N2)C(=O)c2c(F)c(F)c(F)c(F)c21 ; O=C1C(=C2C=Cc3c(ccc(Cc4ccc5c(c4N4C(=O)c6c(F)c(F)c(F)c(F)c6C4=O)C=CC(=C4C(=O)c6c(F)c(F)c(F)c(F)c6C4=O)N5)c3N3C(=O)c4c(F)c(F)c(F)c(F)c4C3=O)N2)C(=O)c2c(F)c(F)c(F)c(F)c21]

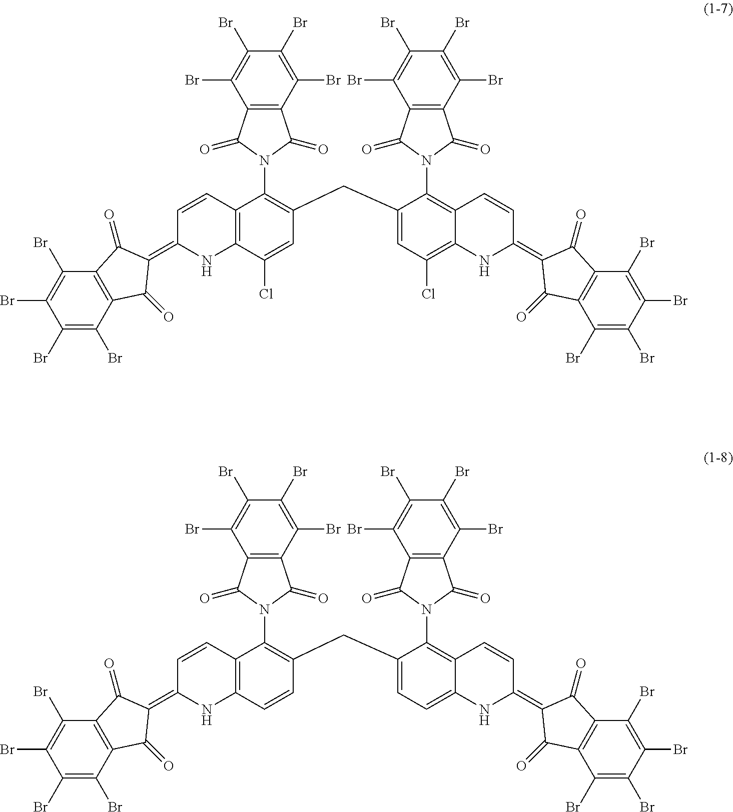 [SMILES: O=C1C(=C2C=Cc3c(c(Cl)cc(Cc4cc(Cl)c5c(c4N4C(=O)c6c(Br)c(Br)c(Br)c(Br)c6C4=O)C=CC(=C4C(=O)c6c(Br)c(Br)c(Br)c(Br)c6C4=O)N5)c3N3C(=O)c4c(Br)c(Br)c(Br)c(Br)c4C3=O)N2)C(=O)c2c(Br)c(Br)c(Br)c(Br)c21 ; O=C1C(=C2C=Cc3c(ccc(Cc4ccc5c(c4N4C(=O)c6c(Br)c(Br)c(Br)c(Br)c6C4=O)C=CC(=C4C(=O)c6c(Br)c(Br)c(Br)c(Br)c6C4=O)N5)c3N3C(=O)c4c(Br)c(Br)c(Br)c(Br)c4C3=O)N2)C(=O)c2c(Br)c(Br)c(Br)c(Br)c21]

The inventive compound may be manufactured by any method and can be appropriately manufactured using a known method. An embodiment of the method for manufacturing the inventive compound will now be described, but the present invention is not limited thereto.

The inventive compound can be prepared by a method including, for example, the following steps I, II, III, and IV.

### <Step I>

According to the method described in J. Heterocyclic. Chem., 30, 17 (1993), 2 to 3 equivalents of crotonaldehyde are added to 1 equivalent of bis-halo aniline, and a reaction is performed in a strong acid in the presence of an oxidizing agent to synthesize a compound represented by Formula (2):

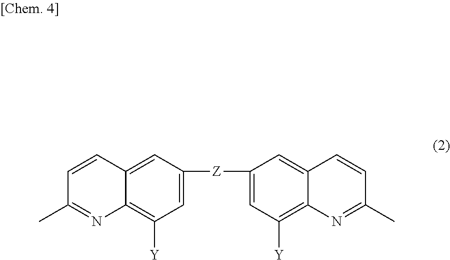

In Formula (2), Y and Z are as described above.

Here, examples of the strong acid include hydrochloric acid, sulfuric acid, and nitric acid.

Examples of the oxidizing agent include sodium iodide, p-chloranil, and nitrobenzene.

Step I can be performed at a reaction temperature of 80° C. to 100° C., preferably 90° C. to 100° C., for a reaction time of 1 to 6 hours, preferably 3 to 6 hours.

### <Step II>

Furthermore, the resulting compound represented by Formula (2) and fuming nitric acid are reacted in the presence of concentrated sulfuric acid. Consequently, a compound represented by Formula (3) can be obtained.

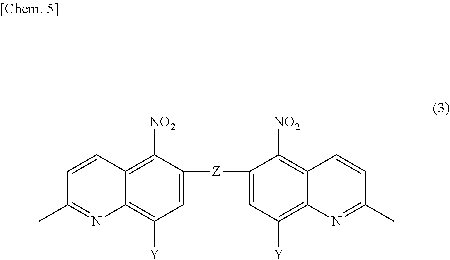

In Formula (3), Y and Z are as described above.

Step II can be performed at a reaction temperature of 40° C. to 70° C., preferably 40° C. to 50° C., for a reaction time of 1 to 3 hours, preferably 1 to 2 hours.

### <Step III>

Furthermore, 6 to 8 equivalents of reduced iron are added to 1 equivalent of the resulting compound represented by Formula (3) to perform a reaction. Consequently, a compound represented by Formula (4) can be obtained.

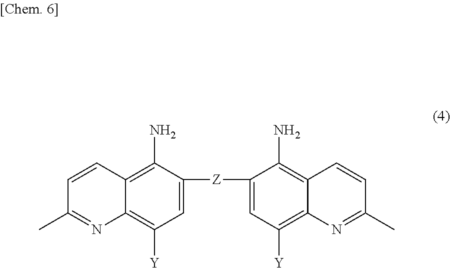

In Formula (4), Y and Z are as described above.

Step III can be performed at a reaction temperature of 60° C. to 80° C., preferably 70° C. to 80° C., for a reaction time of 1 to 3 hours, preferably 2 to 3 hours.

### <Step IV>

Furthermore, for example, according to the method described in Japanese Unexamined Patent Application Publication No. 2013-61622, 4 to 6 equivalents of phthalic anhydride or tetrahalophthalic anhydride are reacted to 1 equivalent of the resulting compound represented by Formula (4) in the presence of an acid catalyst. Consequently, a compound represented by Formula (1) can be obtained.

Here, examples of the acid catalyst include benzoic acid and zinc chloride.

Step IV can be performed at a reaction temperature of 180° C. to 250° C., preferably 210° C. to 250° C., for a reaction time of 1 to 8 hours, preferably 3 to 8 hours.

The inventive compounds may be used alone, or two or more thereof may be appropriately selected to be used in combination.

The inventive compounds are believed to be applicable to a variety of uses. For example, the compounds can be used as coloring agents for a wide range of applications, such as printing inks, paints, colored plastics, toners, ink jet inks, light-shielding members for displays, and seed coloring.

The inventive compound shows properties as organic pigments and can be more suitably used by refinement of pigment particles through, for example, salt milling treatment in some cases. Such treatment may be performed by a publicly known and used method.

The inventive compound may be used in combination with a color material, such as an organic pigment other than the inventive compounds, an organic dye, an organic pigment derivative, for, for example, toning. These coloring materials should be appropriately selected according to the above-described applications, and depending on the application, the inventive compounds may be used alone, or two or more thereof may be appropriately used.

The color material that can be used in combination may be any of known pigments, dyes, and so on.

Depending on the application, examples of the coloring material include azo, disazo, azomethine, anthraquinone, quinophthalone, quinacridone, diketopyrrolopyrrole, dioxazine, benzimidazolone, phthalocyanine, isoindoline, isoindolinone, and perylene pigments, and xanthene, azo, disazo, anthraquinone, quinophthalone, triarylmethane, methine, phthalocyanine, and rhodamine dyes.

Examples of the yellow pigment that can be used in combination with the inventive compound include, in ink application, C.I. Pigment Yellows 3, 12, and 74; in paint application, C.I. Pigment Yellows 74, 83, 109, and 110; and in color filter application, C.I. Pigment Yellows 83, 129, 138, 139, 150, 185, and 231.

In particular, when the inventive compound is used for forming green pixels in color filter application, the inventive compound can be used in combination with, for example, but not limited to, a green pigment, such as C.I. Pigment Greens 1, 2, 4, 7, 8, 10, 13, 14, 15, 17, 18, 19, 26, 36, 45, 48, 50, 51, 54, 55, 58, 59, 62, and 63. When the inventive compound is used for forming green pixels in color filter application, the combination ratio between a green pigment and the yellow pigment of the present invention is, for example, 10 to 150 parts by mass of the yellow pigment for 100 parts by mass of the green pigment.

When the inventive compound is used for forming green pixels, the inventive compound may be used in combination with a blue pigment. Examples of the blue pigment include C.I. Pigment Blues 15, 15:1, 15:2, 15:3, 15:4, and 15:6, and aluminum phthalocyanine derivatives.

Here, examples of the aluminum phthalocyanine derivative include compounds represented by the following Formula (5-1):

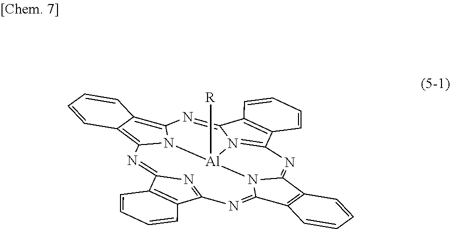

(in Formula (5-1), R is a halogen atom, a hydroxy group, or a group represented by the following Formula (5-2)),

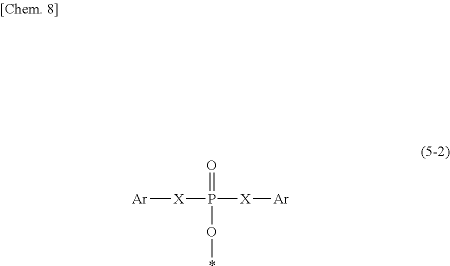

(in Formula (5-2), X is a direct bond or an oxygen atom, Ar is a phenyl group or a naphthyl group, and the asterisk in the formula indicates the bonding site).

Examples of the halogen atom represented by R in Formula (5-1) include fluorine, chlorine, bromine, and iodine atoms. In particular, the halogen atom represented by R is preferably a chlorine atom or a bromine atom.

In Formula (5-1), R is preferably a chlorine atom, a bromine atom, a hydroxy group, or a group represented by Formula (5-2).

In Formula (5-2), X is preferably an oxygen atom.

Preferred examples of the compound represented by Formula (5-1) include hydroxyaluminum phthalocyanine, chloroaluminum phthalocyanine, bromoaluminum phthalocyanine, the compound represented by the following Formula (5-1-1), the compound represented by the following Formula (5-1-2), and the compound represented by the following Formula (5-1-3).

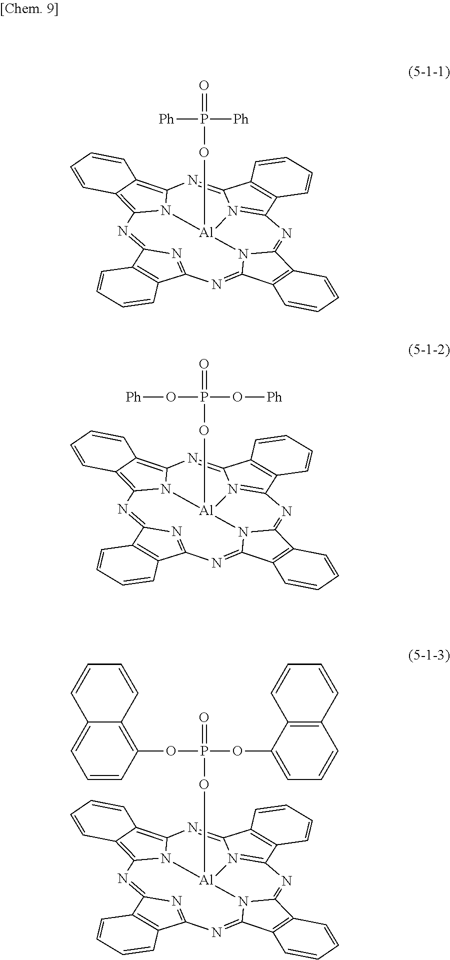

Furthermore, the inventive compound can also be used in combination with a red pigment for forming red pixels in color filter application. Examples of the red pigment include C.I. Pigment Reds 177 and 254.

When the present invention is used for forming a pattern of green pixels of a color filter, a known method can be adopted. Typically, a photosensitive composition for a color filter including the compound of the present invention and a photosensitive resin as essential components can be prepared.

Examples of the method for manufacturing a color filter include a method called photolithography in which a green pattern is prepared by dispersing the inventive compound in a dispersion medium consisting of photosensitive resin, then applying the dispersion onto a transparent substrate, such as a glass substrate, by spin coating, roll coating, ink jetting, or the like, subsequently subjecting the coating film to pattern exposure to UV light through a photomask, and then washing the unexposed portion with, for example, a solvent. This is also the same when the present invention is used for forming a pattern of red pixels of a color filter.

Alternatively, a color filter may be manufactured by forming a pattern of pixels by a method such as an electrodeposition method, a transfer method, a micelle electrolysis method, or a photovoltaic electrodeposition (PVED) method.

A photosensitive composition for a color filter is prepared by, for example, mixing a pigment, a photosensitive resin, a photopolymerization initiator, and an organic solvent for dissolving the resin as essential components. In a general manufacturing method thereof, the pigment is dispersed in the organic solvent optionally using a dispersant to prepare a dispersion, and the photosensitive resin and other components are then added to the dispersant.

As the pigment herein, in the case of preparing green pixels, a pigment formed from the inventive compound and the above-described green pigment or blue pigment can be used. Similarly, in the case of preparing red pixels, a pigment formed from the inventive compound and the above-described red pigment can be used.

Examples of the optionally used dispersant include DISPERBYK (registered trademark) series 130, 161, 162, 163, 170, LPN-6919, and LPN-21116 of BYK-Chemie GmbH; and Efka 46, Efka 47, and Efka 4300 of BASF. In addition, a leveling agent, a coupling agent, a cationic surfactant, or the like can be used in combination.

Examples of the organic solvent include aromatic solvents, such as toluene, xylene, and methoxybenzene; acetate solvents, such as ethyl acetate, butyl acetate, propylene glycol monomethyl ether acetate, and propylene glycol monoethyl ether acetate; propionate solvents, such as ethoxyethyl propionate; alcoholic solvents, such as methanol and ethanol; ether solvents, such as butyl cellosolve, propylene glycol monomethyl ether, diethylene glycol ethyl ether, and diethylene glycol dimethyl ether; ketone solvents, such as methyl ethyl ketone, methyl isobutyl ketone, and cyclohexane; aliphatic hydrocarbon solvents, such as hexane; nitrogen compound-based solvents, such as N,N-dimethylformamide, γ-butyrolactam, N-methyl-2-pyrrolidone, aniline, and pyridine; lactone solvents, such as γ-butyrolactone; and carbamates, such as a mixture of methyl carbamate and ethyl carbamate at 48:52. In particular, suitable organic solvents are water-soluble polar solvents, such as propionate, alcoholic, ether, ketone, nitrogen-compound, and lactone-based solvents.

A dispersion can be prepared by homogeneously stirring and dispersing 300 to 1000 parts by mass of an organic solvent and optionally 100 parts by mass or less of a dispersant and/or 20 parts by mass or less of a quinophthalone derivative for 100 parts by mass of the pigment composition for a color filter of the present invention. Subsequently, a photosensitive composition for color filter pixels can be prepared by adding to this dispersion, 3 to 20 parts by mass of a photosensitive resin for 100 parts by mass of the dispersion, 0.05 to 3 parts by mass of a photopolymerization initiator for 1 part by mass of the photosensitive resin, and optionally an organic solvent and homogeneously stirring and dispersing the mixture.

The pigment composition for a color filter is a mixture of 10 parts by mass of the quinophthalone pigment composition of the present invention, in the case of green pixel application, with 200 parts by mass or less of a green pigment and/or 200 parts by mass or less of a blue pigment, in the case of red pixel application, with 200 parts by mass or less of a red pigment. Another yellow pigment may be further mixed as needed.

Examples of the photosensitive resin that can be used on this occasion include thermoplastic resins, such as urethane resins, acrylic resins, polyamide acid resins, polyimide resins, styrene-maleic acid resins, and styrene-maleic anhydride resins; and photopolymerizable monomers, for example, bifunctional monomers, such as 1,6-hexanediol diacrylate, ethylene glycol diacrylate, neopentyl glycol diacrylate, triethylene glycol diacrylate, bis(acryloxyethoxy)bisphenol A, and 3-methylpentanediol diacrylat, and multifunctional monomers, such as trimethylolpropane triacrylate, pentaerythritol triacrylate, tris(2-hydroxyethyl)isocyanate, dipentaerythritol hexaacrylate, and dipentaerythritol pentaacrylate.

Examples of the photopolymerization initiator include acetophenone, benzophenone, benzyl dimethyl ketal, benzoyl peroxide, 2-chlorothioxanthone, 1,3-bis(4′-azidobenzal)-2-propane, 1,3-bis(4′-azidobenzal)-2-propane-2′-sulfonic acid, and 4,4′-diazidestilbene-2,2′-disulfonic acid.

The thus-prepared photosensitive composition for color filter pixels is subjected to pattern exposure to UV light through a photomask, and the unexposed portion is washed with, for example, an organic solvent or alkaline water to produce a color filter.

### EXAMPLES

The present invention will now be described based on examples, but is not limited thereto. In Examples and Comparative Examples, the terms “part(s)” and “%” are based on mass unless otherwise specified.

### SYNTHETIC EXAMPLES

### Synthetic Example 1

4,4′-Methylene bis(2-chloroaniline) (30.4 g, 114 mmol), p-chloranil (56.1 g, 228 mmol), concentrated hydrochloric acid (29.5 mL), and n-butanol (147 mL) were put in a flask and were stirred at 95° C. for 30 minutes. Crotonaldehyde (19.2 g, 274 mmol) was dropwise added to this mixture, followed by further stirring for 1 hour. The temperature was lowered to 80° C., zinc chloride (15.5 g, 114 mmol) was added thereto in small portions, and THF (100 mL) was then added thereto, followed by stirring for 1 hour while maintaining 80° C. The mixture was allowed to cool to room temperature and was then immersed in an ice bath, and a black powder was collected by filtration under reduced pressure. The resulting black powder was washed with ethanol (500 mL) and was collected again by filtration under reduced pressure. Furthermore, the resulting black powder was transferred to a flask, and ethanol (200 mL) and 10% sodium hydroxide aqueous solution (400 mL) were added thereto, followed by stirring at 80° C. for 1 hour. A powder was collected by filtration under reduced pressure to obtain an intermediate (A) (35.6 g (96.9 mmol), yield: 85%).

¹H-NMR (CDCl₃) δ ppm: 2.81 (s, 6H), 4.24 (s, 2H), 7.34 (d, J=8.0 Hz, 2H), 7.49 (s, 2H), 7.67 (s, 2H), 7.99 (d, J=8.8 Hz, 2H)

¹³C-NMR (CDCl₃) δ ppm: 25.8, 41.1, 123.2, 126.2, 127.8, 130.9, 133.1, 136.3, 137.6, 143.1, 160.0

FT-IR cm⁻¹: 3435, 3054, 3030, 2915, 1603, 1487, 1206

FD-MS: 366 M+

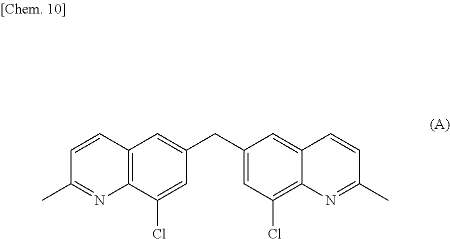

### Synthetic Example 2

The intermediate (A) (4.15 g, 11.3 mmol) prepared in Synthetic Example 1 and concentrated sulfuric acid (7.55 mL) were put in a flask and were stirred at 45° C. for 20 minutes. Subsequently, fuming nitric acid (1.62 mL) was dropwise added to the mixture, followed by stirring for 1 hour while maintaining the temperature. After allowing to cool, iced water (250 mL) was slowly poured into the system. Furthermore, the pH was adjusted to 8 to 9 using 10 wt % sodium hydroxide aqueous solution. The precipitated powder was collected by filtration under reduced pressure and was washed with distilled water (200 mL) and ethanol (100 mL) to obtain a yellowish orange powder of an intermediate (B) (5.13 g (11.2 mmol), yield: 99%).

¹H-NMR (CDCl₃) δ ppm: 2.86 (s, 6H), 4.27 (s, 2H), 7.56 (d, J=8.8 Hz, 2H), 7.62 (s, 2H), 8.08 (d, J=8.8 Hz, 2H)

¹³C-NMR (CDCl₃) δ ppm: 25.7, 32.4, 119.9, 125.6, 127.5, 130.1, 131.1, 137.3, 143.1, 145.9, 162.2

FT-IR cm⁻¹: 3465, 1604, 1530, 1487, 1362

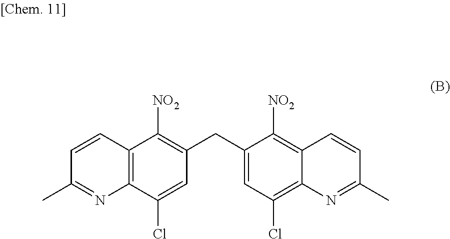

### Synthetic Example 3

The intermediate (B) (5.00 g, 10.9 mmol) prepared in Synthetic Example 2 and ethanol (23.3 mL) were put in a flask and were stirred at room temperature for 10 minutes. Iron (4.88 g, 87.4 mmol) was then added to the system, followed by further stirring at room temperature for 10 minutes. Subsequently, concentrated hydrochloric acid (6.33 mL) was dropwise added thereto, the temperature was raised to 80° C., and stirring was continued for 6 hours. After allowing to cool, the reaction mixture was poured into distilled water (150 ml), and the pH was adjusted to 9 using 10% sodium hydroxide aqueous solution. The precipitated powder was collected by filtration under reduced pressure. Furthermore, the collected powder was sufficiently stirred in ethyl acetate (700 mL), followed by filtration under reduced pressure. The solvent of the resulting filtrate was removed by distillation under reduced pressure to obtain an ocherous powder of an intermediate (C) (1.47 g (3.69 mmol), yield: 34%).

¹H-NMR (CDCl₃) δ ppm: 2.65 (s, 6H), 3.97 (s, 2H), 5.92 (s, 4H), 7.32 (s, 2H), 7.38 (d, J=8.8 Hz, 2H), 8.59 (d, J=8.8 Hz, 2H)

¹³C-NMR (CDCl₃) δ ppm: 25.4, 31.9, 116.8, 117.7, 117.9, 121.0, 131.8, 132.2, 142.0, 143.1, 158.9

FT-IR cm⁻¹: 3476, 3373, 1627, 1605, 1409, 1359, 1250

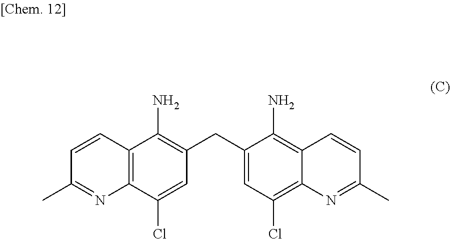

### Synthetic Example 4

Under a nitrogen atmosphere, benzoic acid (14.1 g, 116 mmol) was weighed in a flask and was molten at 140° C. The intermediate (C) (1.44 g, 3.62 mmol) prepared in Synthetic Example 3 and tetrachlorophthalic anhydride (5.53 g, 19.3 mmol) were added to the flask, followed by stirring at 220° C. for 4 hours. After allowing to cool, acetone (300 mL) was added to the reaction solution, followed by stirring for 1 hour. Filtration under reduced pressure was then performed to obtain a yellow powder of a target compound (D) (4.41 g (3.00 mmol), yield: 83%).

FT-IR cm⁻¹: 3449, 1727, 1622, 1536, 1410, 1363, 1308, 1192, 1112, 737

FD-MS: 1467 M+

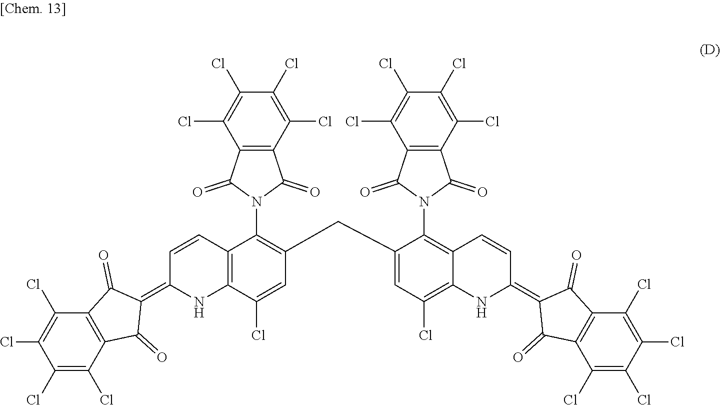 [SMILES: O=C1C(=C2C=Cc3c(c(Cl)cc(Cc4cc(Cl)c5c(c4N4C(=O)c6c(Cl)c(Cl)c(Cl)c(Cl)c6C4=O)C=CC(=C4C(=O)c6c(Cl)c(Cl)c(Cl)c(Cl)c6C4=O)N5)c3N3C(=O)c4c(Cl)c(Cl)c(Cl)c(Cl)c4C3=O)N2)C(=O)c2c(Cl)c(Cl)c(Cl)c(Cl)c21]

### Pigment Formation

The quinophthalone compound (0.500 parts by mass) prepared in the Synthetic Example was ground together with sodium chloride (1.50 parts by mass) and diethylene glycol (0.750 parts by mass). This mixture was then charged into warm water (600 parts by mass), followed by stirring for 1 hour. The water-insoluble matter was collected by filtration and was thoroughly washed with warm water, followed by ventilation drying at 90° C. to form a pigment. The pigment had a particle diameter of 100 nm or less and an average length/width ratio of less than 3.00. The following dispersion test and color filter evaluation test were performed using the resulting quinophthalone compound yellow pigment.

### Manufacturing Example 1

The quinophthalone compound (D) (0.700 parts by mass) was put in a glass bottle. Propylene glycol monomethyl ether acetate (12.8 parts by mass), DISPERBYK (registered trademark) LPN-6919 (manufactured by BYK-Chemie GmbH, 0.467 parts by mass), an acrylic resin solution UNIDIC (registered trademark) ZL-295 (manufactured by DIC Corporation, 0.700 parts by mass), and 0.3-0.4 mm φ Sepr beads (22.0 parts by mass) were added to the bottle, followed by dispersion with a paint conditioner (manufactured by Toyo Seiki Co., Ltd.) for 4 hours to prepare a pigment dispersion. Furthermore, the resulting pigment dispersion (2.00 parts by mass), an acrylic resin solution UNIDIC (registered trademark) ZL-295 (manufactured by DIC Corporation, 0.490 parts by mass), and propylene glycol monomethyl ether acetate (0.110 parts by mass) were put in a glass bottle, followed by shaking to produce a yellow-toning composition.

### Manufacturing Example 2

C.I. Pigment Green 59 (manufactured by DIC Corporation, 2.48 parts by mass) was put in a glass bottle. Propylene glycol monomethyl ether acetate (10.9 parts by mass), DISPERBYK (registered trademark) LPN-6919 (manufactured by BYK-Chemie GmbH, 1.24 parts by mass), an acrylic resin solution UNIDIC (registered trademark) ZL-295 (manufactured by DIC Corporation, 1.86 parts by mass), and 0.3-0.4 mm φ Sepr beads were added to the bottle, followed by dispersion with a paint conditioner (manufactured by Toyo Seiki Co., Ltd.) for 2 hours to prepare a pigment dispersion. Furthermore, the resulting pigment dispersion (4.00 parts by mass), an acrylic resin solution UNIDIC (registered trademark) ZL-295 (manufactured by DIC Corporation, 0.980 parts by mass), and propylene glycol monomethyl ether acetate (0.220 parts by mass) were put in a glass bottle, followed by shaking to produce a green-toning composition.

### Example 1

The yellow-toning composition prepared in Manufacturing Example 1 and the green-toning composition prepared in Manufacturing Example 2 were mixed, and the mixture was applied onto a glass substrate by a spin coater and was then dried. The resulting glass substrate for evaluation was heated at 230° C. for 1 hour, and a green color filter showing each green chromaticity at the time of using a C light source in the color standard for high color reproduction was then produced. As the green chromaticity, (0.210, 0.710) used in Japanese Unexamined Patent Application Publication No. 2013-205581 and (0.230, 0.670) used in Japanese Unexamined Patent Application Publication No. 2011-242425 were used.

### Manufacturing Example 3

A yellow-toning composition was produced as in Manufacturing Example 1 except that C.I. Pigment Yellow 138 (manufactured by BASF) was used instead of the quinophthalone compound (D) and that the amount of the propylene glycol monomethyl ether acetate added before dispersion was changed to 6.42 parts by weight.

### Comparative Example 1

A green color filter was produced as in Example 1 except that the composition prepared in Manufacturing Example 3 was used as the yellow-toning composition, instead of the composition prepared in Manufacturing Example 1.

### Manufacturing Example 4

C.I. Pigment Yellow 150 (manufactured by Sanyo Color Works, LTD., 1.14 parts by mass) was put in a poly bottle. Propylene glycol monomethyl ether acetate (12.0 parts by mass), DISPERBYK (registered trademark) LPN-21116 (manufactured by BYK-Chemie GmbH, 2.84 parts by mass), and 0.3-0.4 mm φ Sepr beads (38.0 parts by mass) were added to the bottle, followed by dispersion with a paint conditioner (manufactured by Toyo Seiki Co., Ltd.) for 4 hours to prepare a pigment dispersion. Furthermore, the resulting pigment dispersion (2.00 parts by mass), an acrylic resin solution UNIDIC (registered trademark) ZL-295 (manufactured by DIC Corporation, 0.490 parts by mass), and propylene glycol monomethyl ether acetate (0.110 parts by mass) were put in a glass bottle, followed by shaking to produce a yellow-toning composition.

### Comparative Example 2

A green color filter was produced as in Example 1 except that the composition prepared in Manufacturing Example 4 was used as the yellow-toning composition, instead of the composition prepared in Manufacturing Example 1.

### Manufacturing Example 5

A yellow-toning composition was produced as in Manufacturing Example 3 except that a quinophthalone monomer (6) synthesized by the method described in Japanese Unexamined Patent Application Publication No. 53-228, instead of C.I. Pigment Yellow 138 (manufactured by BASF).

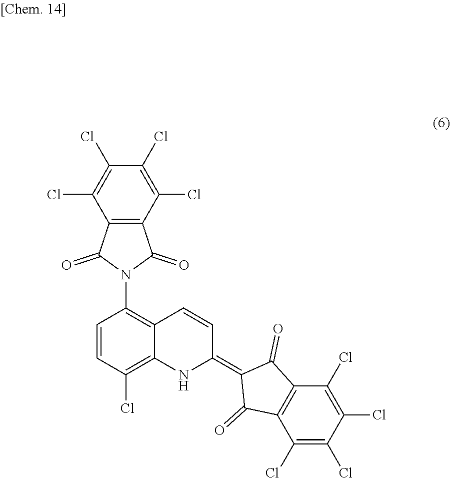 [SMILES: O=C1C(=C2C=Cc3c(N4C(=O)c5c(Cl)c(Cl)c(Cl)c(Cl)c5C4=O)ccc(Cl)c3N2)C(=O)c2c(Cl)c(Cl)c(Cl)c(Cl)c21]

### Comparative Example 3

A green color filter was produced as in Example 1 except that the composition prepared in Manufacturing Example 5 was used as the yellow-toning composition, instead of the composition prepared in Manufacturing Example 1.

### Color Filter Test Example

### Color Filter Characteristic Test

Each of the produced color filters was subjected to measurement of chromaticity and transmission spectrum with a spectrophotometer (manufactured by Hitachi High-Technologies Corporation, U3900/3900H) and to measurement of film thickness (the coloring power is increased with a decrease in the film thickness) with a film thickness meter (manufactured by Hitachi High-Technologies Corporation, VS1000 scanning white light interference microscope). The results are shown in the following Tables 1 and 2.

TABLE 1

(0.210, 0.710) @ C light source

| Color filter | Brightness Y | Film thickness (μm) |
|---|---|---|
| Example 1 | 25.7 | 3.9 |
| Comparative Example 1 | 20.6 | 16.4 |
| Comparative Example 2 | 21.3 | 7.8 |
| Comparative Example 3 | 19.5 | 9.3 |

TABLE 2

(0.230, 0.670) @ C light source

| Color filter | Brightness Y | Film thickness (μm) |
|---|---|---|
| Example 1 | 36.1 | 2.4 |
| Comparative Example 1 | 35.0 | 7.4 |
| Comparative Example 2 | 33.9 | 4.3 |
| Comparative Example 3 | 32.2 | 5.1 |

In Example 1, the brightness was good, and the film thickness was extremely thin, compared to Comparative Example 2 using an existing yellow pigment (C.I. Pigment Yellow 150). This result shows that the yellow pigment of the present invention has high brightness and high coloring power in the color standard of high color reproducibility and is suitable as a yellow pigment for color filters.

In addition, also in comparison with Comparative Example 3 (quinophthalone monomer (6)), in Example 1, the brightness was higher and the film thickness was lower, which demonstrate that the color filter characteristics were significantly improved by dimerization. The quinophthalone monomer (6) is a compound described in Example 2 of Japanese Unexamined Patent Application Publication No. 53-228.

Incidentally, Comparative Example 1 (C.I. Pigment Yellow 138) does not satisfy a practical level due to the low brightness and a very large film thickness. Thus, the inventive compound has notable effects superior to those of existing typical yellow pigments.

### Synthetic Example 5

Under a nitrogen atmosphere, benzoic acid (70.0 g, 573 mmol) was weighed in a flask and was molten at 140° C. The intermediate (C) (2.50 g, 6.29 mmol) prepared in Synthetic Example 3 and 4,5-dichlorophthalic anhydride (7.24 g, 33.3 mmol) were added to the flask, followed by stirring at 220° C. for 6 hours. After allowing to cool, acetone (500 mL) was added to the reaction solution, followed by stirring for 1 hour. Filtration under reduced pressure was then performed to obtain a yellow powder of a target compound (E) (5.92 g (4.96 mmol), yield: 79%).

FT-IR (KBr disk)cm-1: 1789, 1730, 1686, 1624, 1574, 1541, 1409, 1315

FD-MS: 1192 M+

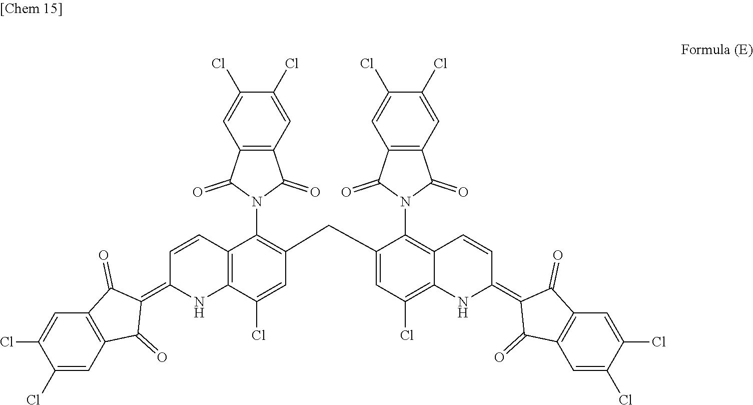 [SMILES: O=C1C(=C2C=Cc3c(c(Cl)cc(Cc4cc(Cl)c5c(c4N4C(=O)c6cc(Cl)c(Cl)cc6C4=O)C=CC(=C4C(=O)c6cc(Cl)c(Cl)cc6C4=O)N5)c3N3C(=O)c4cc(Cl)c(Cl)cc4C3=O)N2)C(=O)c2cc(Cl)c(Cl)cc21]

### Pigment Formation

The quinophthalone compound (the compound represented by Formula (E), 0.500 parts by mass) prepared in Synthetic Example 5 was ground together with sodium chloride (1.50 parts by mass) and diethylene glycol (0.750 parts by mass). This mixture was then charged into warm water (600 parts by mass), followed by stirring for 1 hour. The water-insoluble matter was collected by filtration and was thoroughly washed with warm water, followed by ventilation drying at 90° C. to form a pigment. The resulting pigment had a particle diameter of 100 nm or less and an average length/width ratio of less than 3.00. The following dispersion test and color filter evaluation test were performed using the resulting quinophthalone compound yellow pigment.

### Manufacturing Example 6

A yellow-toning composition was produced as in Manufacturing Example 1 except that the quinophthalone compound (E) prepared in Synthetic Example 5 was used instead of the quinophthalone compound (D).

### Example 2

A green color filter was prepared as in Example 1 except that the yellow-toning composition prepared in Manufacturing Example 6 was used instead of the yellow-toning composition prepared in Manufacturing Example 1.

### Color Filter Test

The green color filter prepared in Example 2 was subjected to measurement of chromaticity and transmission spectrum with a spectrophotometer (manufactured by Hitachi High-Technologies Corporation, U3900/3900H) and to measurement of film thickness (the coloring power is increased with a decrease in the film thickness) with a film thickness meter (manufactured by Hitachi High-Technologies Corporation, VS1000 scanning white light interference microscope). The results are shown in the following

TABLE 3

| | (0.210, 0.710) @ C light source | | (0.230, 0.670) @ C light source | |
|---|---|---|---|---|
| Color filter | Brightness Y | Film thickness (μm) | Brightness Y | Film thickness (μm) |
| Example 2 | 28.8 | 2.9 | 36.8 | 2.0 |

1. A quinophthalone compound represented by Formula (1): 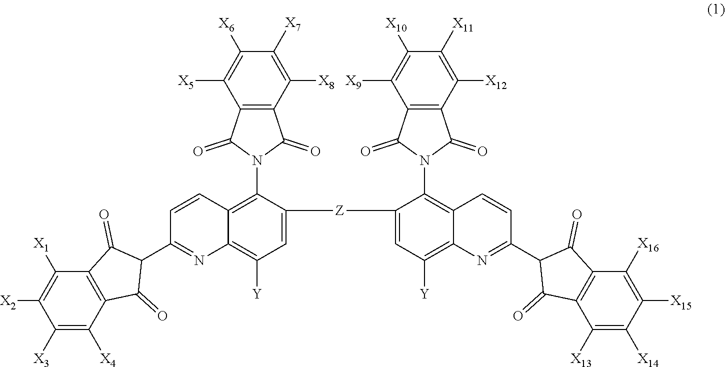
(in Formula (1), X₁ to X₁₆ are each independently a hydrogen atom or a halogen atom, Y is a hydrogen atom or a halogen atom, and Z is a lower alkylene group).

2. The quinophthalone compound according to claim 1, wherein Z is a methylene group.

3. A coloring agent containing the quinophthalone compound according to claim 1.

4. A coloring composition for a color filter containing the coloring agent according to claim 3.
